(19) United States
(12) Patent Application Publication
(10) Pub. No.: US 2023/0223116 A1
(43) Pub. Date: Jul. 13, 2023

(54) METHOD OF CREATING A REMOTE CLINICAL TRIAL WITH A PATIENT'S EXISTING LOCAL PHYSICIAN

(71) Applicant: OncoBay Clinical, Tampa, FL (US)

(72) Inventors: Krystyna KOWALCZYK, Tampa, FL (US); Dannelle Marie PALMER, Tampa, FL (US)

(21) Appl. No.: 18/094,061

(22) Filed: Jan. 6, 2023

Related U.S. Application Data
(60) Provisional application No. 63/297,459, filed on Jan. 7, 2022.

(51) Int. Cl. G16H 10/20

(52) U.S. Cl. CPC G16H 10/20; G16H 40/20

(57) ABSTRACT

The present disclosure relates to a novel method of creating a remote clinical trial to facilitate increased patient participation.

### CROSS REFERENCE

This application claims priority to U.S. Provisional Patent Application No. 63/297,459 filed Jan. 7, 2022, which is incorporated by reference herein.

### TECHNICAL FIELD

Described herein is a novel method for creating, managing, and completing a remote clinical trial site. Aspects of the invention include establishing a site remote from a traditional clinical trial site and facilitating patient recruitment, CRC activities such as scheduling, task management, tracking, and reporting, site management, and completion, all within applicable regulatory requirements and guidance.

### BACKGROUND OF THE INVENTION

Bringing novel medical therapeutics or treatments to market and the general population requires extensive clinical trial testing. These trials are critical to determining safety, efficacy and best patient outcomes. Despite being ranked as the top nation for clinical trial participation, only 5% of the U.S. population participates in clinical trials. (Brennan, 2021). The most significant challenge is finding large, diverse, patient populations that meet the trial criteria, and are willing and able to participate. Many factors play a role in low rates of trial participation, including the lack of accessibility and resources for patients.

For example, the nature of a clinical trial requires recurring site visits to a trial location for ongoing monitoring, testing and evaluation. For patients not located near a trial site, this creates travel and lodging expenses that equate to additional out-of-pocket financial burdens, especially for low-income, rural populations of patients. While out-of-pocket travel expenses may be covered by the Sponsors of some trials, reimbursement for time missed at work is often not covered despite extensive visit schedules. Furthermore, aggressive visit schedules may demand significant time away from family that may not be desirable or practical for many patients.

Furthermore, patients with existing and established personal physicians may be hesitant to involve additional clinicians or leave their existing physician altogether. Where the patient's personal physicians continue to see the patient during the course of the trial, the personal physician must not only be knowledgeable about the trial and the patient's health, but to be able to communicate with the clinical trial and staff regularly, and understand the impact of their treatment. Such additional considerations may be burdensome for both the patient and the patient's personal physician, hindering participation.

Thus, there exists a need to address lack of patient participation in clinical trials.

### SUMMARY OF THE INVENTION

One embodiment described herein is a method of creating a remote clinical trial to recruit one or more patients to an existing clinical trial comprising: a) identifying one or more patients; b) identifying one or more local physicians having a clinic local to the one or more patients; c) preparing the one or more local physicians and the respective clinic for research qualification; and d) facilitating conduct of the clinical trial for the one or more local physicians. In another aspect of the method, identifying one or more patients comprises matching each patient's diagnosis and treatment requirements to a clinical trial. In another aspect, the method further comprises qualifying each patient for a clinical trial. In another aspect, identifying one or more local physicians comprises: a) selecting each patient's personal physician to become the local physician; b) providing each patient with technology and resources to identify the local physician who is not the patient's personal physician; or c) providing each patient's personal physician with technology and resources to identify the local physician.

In another aspect of the method, identifying one or more local physicians further comprises approving and qualifying at least one local physician to serve as an Investigator of the clinical trial by a Sponsor of the clinical trial. In another aspect, approving and qualifying the at least one local physician comprises complying with 21 CFR § 312 and the International Council for Harmonization for Better Health/Good Clinical Practices (ICH E6 (R2)). In yet another aspect, preparing the at least one local physician comprises executing a contract with each local physician to serve as a Principal Investigator or a sub-Investigator for the remote clinical trial. In one aspect, the contract for the Principal Investigator comprises a trial budget comprising an amount of payment to the local physician and a budget for support services for the local physician. In another aspect, the contract for the sub-Investigator comprises a trial budget comprising an amount of payment to the local physician, services a Principal Investigator will provide to the local physician, services the local physician will provide, and a budget for support services for the local physician.

In another aspect, preparing the clinic for research qualification comprises complying with 21 CFR § 312 and the International Council for Harmonization for Better Health/Good Clinical Practices (ICH E6) and comprises: a) obtaining regulatory approvals; b) providing training to staff; c) creating standard operating procedures for the clinic to maintain research qualification; d) providing an electronic Investigator Site File; e) obtaining equipment; or f) obtaining technology to enable a remote clinical trial.

In another aspect, the regulatory approvals comprise collecting and maintaining regulatory documents. In one aspect, the regulatory documents are transmitted or delivered to the regulatory authority. In another aspect, the staff may be on site at the clinic or may work remotely. In yet another aspect, the technology comprises one or more of telemedicine, cloud computing, video conferencing, electronic learning management systems, electronic signature software, data privacy and security software.

In one aspect, facilitating conduct of the clinical trial for the local physician comprises preparing subject level specific training for the site and visit-by-visit walk-through guides for the purposes of ensuring just-in-time training for the local physician and local clinic personnel, as well as for ensuring appropriate documentation of procedures conducted for the purposes of ensuring ongoing compliance with ICH E6 (R2) Sections 4.1.2 and 4.1.5. In another aspect, facilitating conduct of the clinical trial for the local physician further comprises providing ongoing remote support for the local physician. In another aspect, facilitating conduct of the clinical trial for the local physician further comprises one or more of facilitating data entry, answering study-related questions, regulatory document management, and trial management to ensure continued compliance with 21 CFR § 312 and the International Council for Harmonization for Better Health/Good Clinical Practices (ICH E6). In another aspect, facilitating conduct of the clinical trial for the local physician comprises one or more of ongoing facilitation of clinical site activities conducting the trial until the patient completes the trial or trial follow-up, or discontinues the trial.

In one aspect, the completion or discontinuation of the trial comprises one or more of facilitating closure of the clinic and return of equipment or technology.

### BRIEF DESCRIPTION OF THE DRAWINGS

FIG. 1: Describes a flow chart depicting an example method of creating a “pop-up” or remote clinical trial site.

### DETAILED DESCRIPTION OF THE DISCLOSURE

The following description and examples illustrate embodiments of the present disclosure in detail. It is to be understood that the present disclosure is not limited to the particular embodiments described herein and as such may vary. Those of skill in the art will recognize that there are variations and modifications of the present disclosure, which are encompassed within its scope.

All publications, mentioned in this specification are herein incorporated by reference to the same extent as if each individual publication, patent, or patent application was specifically and individually indicated to be incorporated by reference.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “some aspects,” “an embodiment,” “an aspect,” “one embodiment,” “one aspect” or “other embodiments” or “other aspects” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “just-in-time training” means training delivered on demand and as needed. Training is not expected to occur prior to the demand.

As used herein, the term “local to a patient” or “local to the patient” means in close physical proximity to a patient. For example, local to a patient means, within walking distance to a patient, within driving distance to a patient, within about 1 mile to about 100 miles of where a patient resides, within about 10 miles to about 90 miles, within about 20 miles to about 80 miles, within about 30 miles to about 70 miles, within about 40 miles to about 60 miles, within about 50 miles, within about 1 mile to about 20 miles, within about 5 miles to about 15 miles, within about 10 miles. As used herein, the term “local physician” means a physician that is local to a patient.

As used herein the term “qualifying the local physician” or “preparing the clinic for research qualification” means ensuring the local physician and/or local physician's clinic comply with the specific regulatory body governing clinical trials in the country in which the trial is being conducted. For example, in the United States, this may entail complying with 21 CFR § 312 and/or the U.S. Food and Drug Administration's federal guidance for good clinical practice as described in International Council for Harmonization for Better Health/Good Clinical Practices (ICH E6 (R2)).¹ In some aspect, this may mean complying with the regulatory body governing clinical trials in another country. ¹21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

As used in herein a “remote clinical trial” or “personal clinical trial” means an alternative location to an existing clinical trial that may be set up to facilitate patient participation in the existing clinical trial. In some aspects, the remote clinical trial or personal clinical trial may be physically located in close proximity to a patient that qualifies for an existing clinical trial. In some aspects, the remote clinical trial or personal clinical trial may be physically located in close proximity to a physician that is treating a patient that qualifies for participation in the existing clinical trial. This is believed to be a novel aspect of the present disclosure and, as such, is intended to encompass a variety of physical variants including but not limited to a patient located remote from a traditional clinical trial site, or a physician treating one or more patient, where either the one or more patients or the physician is located remote from a traditional clinical trial site, or one or more physician treating one or more patient is collated to create a collective remote clinical trial remote from a traditional clinical trial site.

As used herein “Sponsor”, or “site Principal Investigator” may determine the medical or clinical procedures (or activities) to be performed based on the clinical trial/study protocol. Such procedures may include x-rays, CT scans, blood draws, reading of vital signs, office consultations—likely any procedure for which there is a medical procedure or CPT code. Procedures without a CPT code, such as filling out questionnaires, study assessments, and other clinical study activities, may also be included.

As used herein the term “subject visit-level specific training” means that rather than training a local physician on the whole clinical trial study protocol, they are trained to conduct each visit with the patient just prior to that visit. Doing so ensures they are not trained on more than what they may need (or trying to absorb too much information) and they have the information when they do need it.

The term “visit-by-visit walk-through guides” means subject visit-specific checklists that tell a local physician in writing what procedures to conduct, in what order, and what data to capture for each subject visit.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

To address the lack of patients participating in clinical trials, described herein is a novel method of building a “pop-up” clinical trial site (also referred to herein as a “remote clinical trial site”), at a location that is convenient, accessible and comfortable for one or more patients and/or a patient's physician(s) or another local physician. Building the clinical trial site around the patient and/or their local physician or another physician local to the patient facilitates increased enrollment in the trial, diversity, reduces patient burdens and progresses the clinical trial study. Accordingly, the present disclosure contemplates a combination of steps that operates in a non-conventional way to ensure that a patient that is not physically located in close proximity to a clinical trial has the ability to participate. The method described herein is thus more than merely organizing and data gathering, but instead sets up a unique combination of steps to address a unique problem associated with the lack of participation in, or access to, a clinical trial.

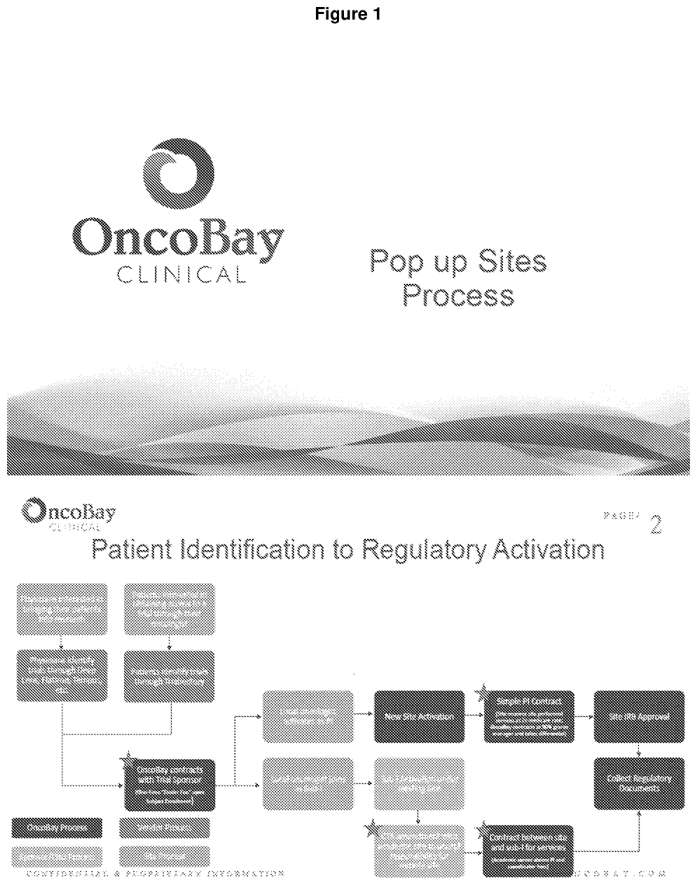

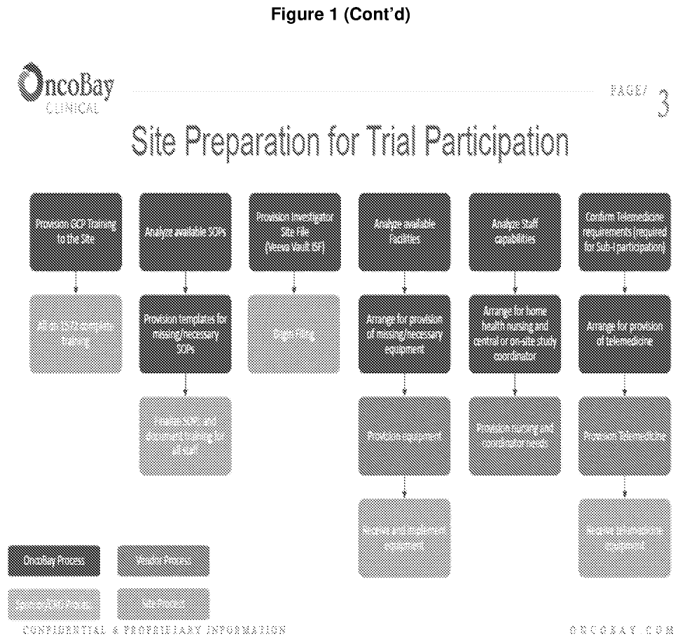

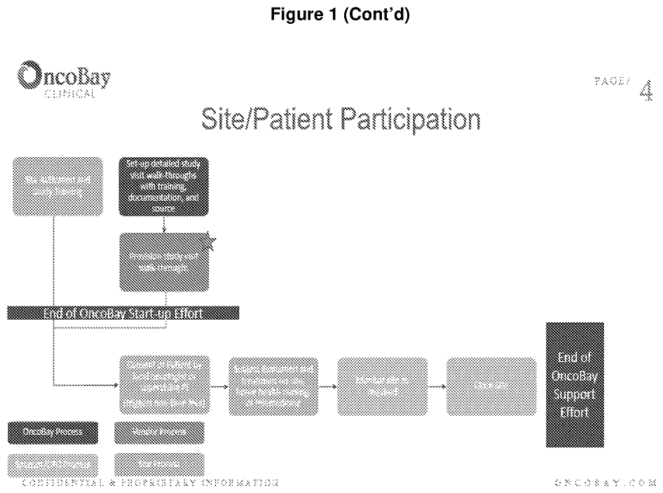

Thus, one embodiment described herein is a method of creating a remote clinical trial to recruit one or more patients to an existing clinical trial comprising: a) identifying one or more patients; b) identifying one or more local physician(s) having a clinic local to the patient; c) preparing the local physician and the clinic for research qualification; and d) facilitating conduct of the clinical trial for the local physician. FIG. 1 describes a flow chart depicting an example method of creating a “pop-up” or remote clinical trial site.

The remote clinical trial may comprise any type of clinical research or study requiring patient participation, including for example, treatment research, prevention research, diagnostic research, screening research, quality of life research, genetic studies or epidemiological studies. Furthermore, the clinical trials may be in any phase, including phase I, phase II, phase III or phase IV trials. Any type of clinical trial may be set up as a remote clinical trial. The remote clinical trial offers an alternative location to an existing clinical trial that may be set up to facilitate patient participation in the existing clinical trial. In some aspects, the remote clinical trial may be physically located in close proximity to a patient that qualifies for an existing clinical trial. In some aspects, the remote clinical trial may be physically located in close proximity to a physician that is treating a patient, or another physician in close proximity to the patient that qualifies for participation in the existing clinical trial. The remote clinical trials described herein may be set up in any city, state in the United States, or in any country so long as the trial complies with the regulatory authority governing the clinical trial in the country in which the trial is taking place. Thus, the remote clinical trials described herein may be created in, for example, the U.S., France, Canada, China, Germany, the U.K., or South Korea.

In order to create the remote clinical trial, a patient meeting the clinical trial criteria may be identified, and the patient may not be located within close proximity to an existing clinical trial. The patient may be identified in variety of ways, including for example, self-identification, identification by the patient's personal physician, employer, local hospital, healthcare clearing house, contract research organization (CRO), trial finder databases, patient support groups or a patient recruitment service, or clinicaltrials.gov. Identification may also be made by a review of protected health information (PHI) in an any electronic media form, such as a database or website with patient health information available to match patients to clinical trials. Any source of patient identification is acceptable, so long as the patient meets the clinical trial criteria and patient privacy regulations in that jurisdiction are honored throughout the process. Thus, recruitment may be based on a variety of patient criteria, depending on the purpose of the clinical trial. For example, for clinical trials in which potential cancer therapies are tested, patients with an oncology diagnosis would be appropriate candidates. Alternatively, patients with cardiovascular issues may be suitable for participation in clinical trials designed to test cardiovascular therapies. For trials in which screening research is performed, a patient with a family history of certain disease, or having a specific genetic makeup, may be suited for participation. Furthermore, patients should also meet all other requirements of a clinical trial, including for example, demographic requirements such as specific age, race, sex or ethnicity. Thus, in one aspect described herein, identifying a patient comprises matching a patient's physical or mental health diagnosis, family history, genetic profile, disorder, treatment or demographic profile to requirements of a clinical trial. In another aspect, identifying a patient comprises matching a patient's diagnosis and treatment requirements to a clinical trial.

Once a patient is identified, the patient is contacted to confirm their interest in participating in an existing clinical trial, at a remote clinical trial site. If a potential patient agrees, a local physician having a clinic local to the patient may be identified. The present disclosure contemplates using a physician and/or a physician's office local to the patient to serve as the remote clinical trial site. The physician may be the patient's current local, personal physician or another local physician willing to serve as a clinician for a remote clinical trial. Both the patient and physician may be provided with technology and resources to identify another local physician should an initially identified physician be unwilling or unable to participate. Exemplary technology and resources for finding a suitable physician include referral services, physician directories, healthcare and medical doctor databases, online physician referral websites, government agency referral resources, patient search engines—either clinical trial specific or through their own physician group. Thus, in one aspect, identifying a local physician comprises: a) selecting the patient's personal physician to become the local physician; b) providing the patient with technology and resources to identify the local physician who is not the patient's personal physician; or c) providing the patient's personal physician or another physician with the technology and resources to identify the local physician.

Should no local physician be identified or be willing to participate, patients may be asked if they are willing to participate in the next closest remote clinical trial site. If the patient agrees, the patient is directed to the next closest remote clinical trial site for enrollment (assuming one exists, and if not, the process for creating a remote clinical trial site begins again with identifying a possible local physician). However, should a local physician agree to service a remote clinical trial site for a local patient, consents from both the patient and the local physician are obtained, and the local physician is then subject to an approval and qualification process.

In order for a local physician to service a remote clinical trial site, the local physician must be approved and qualified in accordance with the regulatory authority governing clinical trials in the country in which the trial is taking place. For example, in the U.S., a local physician would need to be approved and qualified in accordance with 21 CFR § 312 and the International Council for Harmonization for Good Clinical Practices (ICH E6 (R2)).¹. In Europe, a local physician and a clinical trial would need to comply with the all the requirements set forth by the European Medicines Agency (EMEA) and Commission Directive 2001/20/EC, Directive 2005/28/EC and Directive 2003/94/EC, providing rules on Good Clinical Practice (GCP). ¹21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

Thus, under U.S. law, qualification of the local physician comprises qualifying the local physician to become a Principal Investigator for the remote clinical trial, or work under the supervision of a Principal Investigator or another study investigator, of the existing clinical trial as a sub-Investigator.

This further comprises that the local physician agree to all protocols, training, records requirements, compliance requirements, and investigator obligations, required by the governing law.

Thus, in one aspect, identifying a local physician further comprises approving and qualifying the local physician to serve as an Investigator of the clinical trial by a Sponsor of the clinical trial.

In another aspect, identifying the local physician comprises approving and qualifying the local physician comprises complying with 21 CFR § 312 and the International Council for Harmonization for Better Health/Good Clinical Practices (ICH E6 (R2)).

Once a physician has agreed to take on the obligations of becoming a Principal Investigator or work as a sub-Investigator, the local physician's credentials may be reviewed and collected, including for example, a resume and medical license information. The local physician may execute a series of written contracts and consents agreeing to take on all obligations of servicing the remote clinical trial, setting physician payment, setting and agreeing to a trial budget based on the schedule of assessments in the study protocol, support services provided by a Principal Investigator to the local physician, or support services the local physician may require from others, and other required budget related information. All documentation required for compliance may be provided to the Sponsor of the trial and/or the Principal Investigator under the governing regulatory law for approval.

Thus, in one aspect, preparing the local physician comprises executing a contract with the local physician to serve as a Principal Investigator or a sub-Investigator for the remote clinical trial. In another aspect, the contract for the sub-Investigator comprises a trial budget comprising an amount of payment to the local physician, services a Principal Investigator will provide to the local physician, services the local physician will provide, and a budget for support services for the local physician.

In addition to identifying patients and physicians for a remote clinical trial site, physicians that agree to service a remote clinical trial site may also need a local clinic to serve as the primary location to see patients enrolled in the remote clinical trial. The location of the remote clinical trial site should be local to a patient and/or the local physician servicing the trial, and may be able to reach easily and on a regular basis, or local to a patient and/or physician. As used herein, the term “local to a patient” or “local to the patient” means in close physical proximity to a patient. For example, local to a patient means, within walking distance to a patient or within driving distance to a patient. Exemplary distance includes, but is not limited to, about 1 mile to about 100 miles from where a patient and/or physician resides, or from where a physician works; within about 10 miles to about 90 miles from where a patient and/or physician resides, or from where a physician works; within about 20 miles to about 80 miles from where a patient and/or physician resides, or from where a physician works; within about 30 miles to about 70 miles from where a patient and/or physician resides, or from where a physician works; within about 40 miles to about 60 miles from where a patient and/or physician resides, or from where a physician works; within about 50 miles from where a patient and/or physician resides, or from where a physician works; within about 1 mile to about 20 miles from where a patient and/or physician resides, or from where a physician works; within about 5 miles to about 15 miles from where a patient and/or physician resides, or from where a physician works; within about 10 miles from where a patient and/or physician resides, or from where a physician works.

Clinics may be the local physician's location of private practice, or another clinic or medical facility that agrees to allow its location to serve as a clinical trial site. Like physicians, clinics must also be approved for research qualification according to the regulatory body governing clinical trials in a particular country. For example, in the U.S., clinics may be qualified to serve as a clinical trial site in accordance with 21 CFR § 312 and the International Council for Harmonization for Good Clinical Practices (ICH E6). In Europe, remote clinical trial sites must operate in accordance the requirements set forth by the European Medicines Agency (EMEA) and Commission rules on Good Clinical Practice (GCP).

This may require clinics serving as remote clinical trial sites to obtain regulatory approvals, provide clinic staff with requisite training on site (or remote training), creating standard operating procedures; obtain equipment and other technology required to enable a remote clinical trial to conduct the trial.

In some aspects, qualifying and setting up a clinic to serve as a remote clinical trial site may require contracting with vendors that may provide the support services, equipment, technology, supplies and resources required to set up the location. For example, a remote clinical trial site may require good clinical practice (GCP) training for the staff conducting the trials. Training may need to be provided on site (or remote) and documentation of training and certifications may need to be recorded and retained. Anything required to facilitate training may need to be provided by a vendor, including for example, equipment and technology, such as telemedicine equipment, computers, laptops, cameras, binders for storing paper information, secured file cabinets for records storage and retention, freezers, refrigerators, locked cabinets for storing study drugs, thermometers. Furthermore, some staff may work remotely, such as the Sponsor or Principal Investigator or their staff, who may be physically located at the site of the existing clinical trial, or another remote clinical trial site. Any equipment required to facilitate remote work or telemedicine may be required and procured by the vendors to enable the remote clinical trial site to be functional. Exemplary technology equipment may include telemedicine, directed electronic data capture systems, cloud computing, video conferencing, electronic learning management systems, electronic signature software, data privacy and security software. Exemplary technology may also require setting up of an acceptable electronic Investigator Site File (eISF) system as required by the Sponsor, or patient information databases.

Another example of when specialized equipment may be required is equipment needed to conduct the trial itself, such as medical imaging machines (X-ray, MRI, CT), durable medical equipment such as hospital beds, specialized physical therapy equipment, treatment equipment such as infusion pumps, surgical machines, lasers, diagnostic scopes, specialized blood draw equipment, autoclaves, and pharmaceuticals. Any equipment that is not already available for use or present at the site of the remote clinical trial and may be needed according to the clinical trial study protocol, may be procured by vendors to prepare the site, or obtained from the Sponsor.

Thus, in one aspect, preparing the clinic for research qualification comprises complying with 21 CFR § 312 and the International Council for Harmonization for Better Health/Good Clinical Practices (ICH E6) and comprises a) obtaining regulatory approvals; b) providing training to staff; c) creating standard operating procedures for the clinic to maintain research qualification; d) providing an electronic Investigator Site File; e) obtaining equipment; or f) obtaining technology to enable a remote clinical trial. In one aspect, the regulatory approvals comprise collecting and maintaining regulatory documents. In another aspect the staff may be on site at the remote clinical trial or may work remotely. In another aspect, the technology comprises telemedicine, cloud computing, video conferencing, electronic learning management systems, electronic signature software, data privacy and security software.

Remote clinical trial sites may be monitored at all times and throughout the clinical trial for site and staff compliance, training, inventory and resources to ensure the site and staff are in compliance, fully equipped and capable of conducting the trial study at the remote clinical trial location. Furthermore, the remote clinical trial sites and the local physician servicing the site, may be monitored and may need to remain in constant communication with the Sponsor and/or a Principal Investigator during the course of the clinical trial to ensure all standard operating procedures and study protocols are communicated and followed; patient visits, patient information, data are being logged, collected and carried out according to the clinical trial protocol. Communication may require the need for telemedicine and access to websites, databases and software, such as for example, Genius ENGAGE (eICF) and/or Genius ROSA and Trial Navigator, in order to facilitate communication, data collection and patient monitoring. The site should be set up to include access to all such technology during the entire duration of the trial.

Thus, in one aspect described herein, facilitating conduct of the clinical trial for the local physician comprises ongoing facilitation of clinical site activities conducting the trial until the patient completes the trial or trial follow-up, or discontinues the trial. In another aspect, facilitating conduct of the clinical trial for the local physician comprises preparing subject visit-level specific training for the site and visit-by-visit walk-through guides for the purposes of ensuring just-in-time training for the local physician and local clinic personnel, as well as for ensuring appropriate documentation of procedures conducted for the purposes of ensuring ongoing compliance with ICH E6 (R2) Sections 4.1.2 and 4.1.5. In another aspect, facilitating conduct of the clinical trial for the local physician further comprises providing ongoing remote support for the local physician. In another aspect, facilitating conduct of the clinical trial for the local physician further comprises facilitating data entry, answering study-related questions, regulatory document management, and trial management to ensure continued compliance with 21 CFR § 312 and the International Council for Harmonization for Better Health/Good Clinical Practices (ICH E6). In another aspect, facilitating conduct of the clinical trial for the local physician comprises ongoing facilitation of clinical site activities conducting the trial until the patient completes the trial or trial follow-up, or discontinues the trial.

Once a patient completes a trial, the Sponsor and Principal Investigator may verify that the trial is complete according to the study protocol, all data obtained is collected, recorded and transferred to the Sponsor. At this point, the clinical trial site may be closed. Any equipment or technology procured specifically for the remote clinical trial location may be returned or retained as needed. Local physicians conducting the trial may have remaining or ongoing obligations that they may need to continue complying with, which may be supported through ongoing training and the availability of additional facilities, equipment, and expertise support as needed. In another aspect, completion or discontinuation of the trial, comprises facilitating closure of the clinic and return of equipment or technology.

It is further understood that any of the methods described herein may be computer implemented methods and executed on a standalone computer, on a network, or a mobile phone application, for example, over the Internet as a cloud-based service or hosted service, which may be accessed through a standard web service application programming interface (API).

Aspects of the present disclosure may be embodied in the form of a system, a computer program product, or a method. Similarly, aspects of the present disclosure may be embodied as hardware, software or a combination of both. Aspects of the present disclosure may be embodied as a computer program product saved on one or more computer-readable media in the form of a computer-readable program code embodied thereon.

For example, the computer-readable medium may be a computer-readable signal medium or a computer-readable storage medium. A computer-readable storage medium may be, for example, an electronic, optical, magnetic, electromagnetic, infrared, or semiconductor system, apparatus, or device, or any combination thereof.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device.

Computer program code in embodiments of the present disclosure may be written in any suitable programming language. The program code may execute on a single computer, or on a plurality of computers. The computer may include a processing unit in communication with a computer-usable medium, wherein the computer-usable medium contains a set of instructions, and wherein the processing unit is designed to carry out the set of instructions.

### EXAMPLES

### Example 1: Standard Operating Procedure for Identifying a Patient

Example 1 is exemplary Standard Operating Procedure (SOP) for use in identifying a patient for recruitment to a remote clinical trial. The standard operating procedure outlines the various steps required to execute the method described herein for identifying a patient.

| Document | | | | Department/ | Central Project |
|---|---|---|---|---|---|
| Type: | Standard Operating Procedure | | | Owner: | Operations |
| Title: | Patient Matching | | | Document | xx |
| | | | | Number: | |
| Review | Every 2 | Effective | DD-Mon- | Version | 1.0 |
| Frequency: | years | Date: | YYYY | Number: | |

### Purpose

This Standard Operating Procedure (SOP) describes the process of identifying a patient for the purpose of creating a remote clinical trial environment at a location where the patient is already receiving care.

### Scope

This SOP applies to all OncoBay employees and contractors involved in patient identification for personal clinical trial environments. Vendors qualified for patient identification by QA011 Selection and Qualification of Contract Service Providers will follow their own processes for patient identification, and this SOP will connect the identified patient to an existing clinical trial.

Identifying a patient comprises matching a patient's oncology diagnosis and treatment requirements to an existing clinical trial. This process further comprises qualifying the patient for the clinical trial.

### Definitions and Abbreviations

| Term | Definition |
|---|---|
| Notifier | The patient, physician, patient advocate, or patient recruitment service that notifies |
| | OncoBay of an interested patient, physician, and clinical trial. |
| Protected | PHI is information that (i) is created or received by a health care provider, |
| Health | employer or health care clearing house; (ii) identifies an individual or reasonably |
| Information | could identify him/her; (iii) relates to an individuals' physical or mental health, |
| (PHI) | health care services provided to the individual, or payment for such health care |
| | services; and (iv) is transmitted by, or maintained in, any electronic media or other |
| | form. PHI does not apply to information that has been de-identified. |
| Network | This is a designated role that may be performed by a Director, Manager, |
| Manager | Lead, or Specialist within Network Operations at OncoBay Clinical. |

### Procedure

Patient Identification

| Step | Action | Responsible Party |
|---|---|---|
| A.1 | Upon notification of a potential patient match, contact the | Network Manager |
| | Notifier to confirm patient interest. | (NM) |
| | NOTE: Potential patient matches may be identified | |
| | through trial finder databases published by patient | |
| | advocacy groups and other patient support groups, | |
| | clinicaltrials.gov, or other patient matching services. | |
| | Confirm that there is an identifiable patient, physician, site | |
| | location, and clinical trial. If all elements are present, skip | |
| | to Step A.3. If the notifier is a patient or patient caregiver | |
| | and the physician is not identified, proceed to step A.2. | |
| A.2 | Obtain contact information for the patient's treating | NM |
| | physician and contact the physician to confirm if that | |
| | physician is willing to participate. If the treating physician | |
| | or another physician at the patient's existing treatment | |
| | center is willing to participate, proceed to Step A.4. | |
| A.3 | If the patient's existing treatment center is not willing to | NM |
| | participate, identify the nearest active site on the trial using | |
| | clinicaltrials.gov or another available trial reporting | |
| | database (e.g., SiteTrove by Informa). Confirm if the | |
| | patient is willing to travel to the site, and then contact the | |
| | site to confirm if the site will accept assignment of the | |
| | patient. If the site will accept assignment of the patient, | |
| | provide the patient with the site's contact information and | |
| | follow up with the site to ensure the connection is made. | |
| | The process ends here. If the patient is not willing to travel | |
| | to the site or the site is not willing to accept assignment, | |
| | repeat this step until an acceptable site connection is | |
| | made. | |
| A.4 | Provide the Notifier with XXX “Consent Form” for patient | NM |
| | and physician to consent to the Pop-Up Site process. | |
| | Attach the full record of information of the clinical trial from | |
| | clinicaltrials.gov. | |
| A.5 | Collect from the Notifier XXX “Consent Form”. If patient | NM |
| | eligibility is unclear, collect from the Notifier relevant patient | |
| | records and ensure that the protected health information | |
| | (PHI) and all patient identifiers are redacted. If there is | |
| | information requiring redaction, redact the information as | |
| | required using appropriate software or by printing, | |
| | redacting, and then scanning the information. Verify | |
| | eligibility against clinicaltrials.gov. | |
| A.6 | Collect from the Notifier the curriculum vitae (CV) of the | NM |
| | proposed local physician. | |
| | Note: The CV does not require alignment with SA001_WP06 | |
| | Collection and Processing of Essential Documents at this point in | |
| | the process, but should have a broad overview of the physician's | |
| | experience. | |
| A.7 | Upon receipt of the signed form and confirmation that the | NM |
| | relevant patient records match trial eligibility, contact the study | |
| | contacts listed on clinicaltrials.gov to notify them of patient | |
| | interest. Present XXXX “Marketing Materials” to offer | |
| | OncoBay's services in activating the local physician. | |
| A.8 | If the study contact accepts OncoBay's services, proceed to | NM |
| | Section B. If the study contact declines OncoBay's services, | |
| | attempt to identify an alternate trial and proceed to step A.9. | |
| A.9 | If an alternate trial is identified, discuss with the Notifier to | NM |
| | confirm if the patient and physician may be interested in another | |
| | trial. If potential interest is confirmed, return to step A.2. | |

Trial Sponsor Contracting

| Step | Action | Responsible Party |
|---|---|---|
| B.1 | Provide the study contact with XXX “Short Form Contract” | NM |
| | and the CV of the local physician. Request confirmation of | |
| | patient acceptance and identification of appropriate legal | |
| | contacts within 24 hours. | |
| B.2 | Upon confirmation of patient eligibility, provide legal | NM |
| | contact information (if received) or study contact | |
| | information to legal@oncobay.com. | |
| B.3 | Execute the Short Form Contract in accordance with “SOP | Legal |
| | Sponsor Contracting”. | Representative |

### Example 2: Standard Operating Procedure for Identifying a Local Physician

Example 2 describes an exemplary Standard Operating Procedure (SOP) for use in identifying a local physician having a clinic local to a patient. The standard operating procedure outlines the various steps required to execute the method described herein for identifying a local physician.

| Document | Standard Operating Procedure | | | Department/ | Central Project |
|---|---|---|---|---|---|
| Type: | | | | Owner: | Operations |
| Title: | Activating a Local Physician | | | Document | xx |
| | | | | Number: | |
| Review | Every 2 | Effective | DD-Mon- | Version | 1.0 |
| Frequency: | years | Date: | YYYY | Number: | |

### Purpose

This Standard Operating Procedure (SOP) describes the process of identifying a local physician having a clinic local to a patient for the purpose of creating a personal clinical trial environment at a location where the patient is already receiving care.

### Scope

This SOP applies to all OncoBay employees and contractors involved in local physician identification for personal clinical trial environments. Identifying a local physician can occur through the following means:
Selecting the patient's personal physician to become the local physician;
providing the patient with technology and resources to identify the local physician who is not the patient's personal physician; or
providing the patient's personal physician with technology and resources to identify the local physician.

When the patient's personal physician will become the local physician, this may include approving and qualifying the local physician to serve as an Investigator of the clinical trial by a Sponsor of the clinical trial in accordance with 21 CFR § 312 and the International Council for Harmonization for Good Clinical Practices (ICH E6 (R2)).¹. Alternatively, this may comprise executing a contract with the local physician to serve as a Principal Investigator or a sub-Investigator for a personalized clinical trial environment. ¹21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

Providing the patient or the patient's personal physician with technology and resources to identify the local physician are beyond the scope of this SOP.

### Definitions and Abbreviations

| Term | Definition |
|---|---|
| Network | This is a designated role that can be performed by a |
| Manager | Director, Manager, Lead, or Specialist within Network |
| | Operations at OncoBay Clinical. |

### Procedure

Activating a Local Oncologist as a Subinvestigator Under an Existing Principal Investigator

| | | Responsible |
|---|---|---|
| Step | Action | Party |
| A.1 | Confirm with the Notifier contact information for the | Network |
| | patient's physician. | Manager |
| | | (NM) |
| A.2 | Confirm with the patient's physician if they or another | NM |
| | local physician will act as the Principal Investigator | |
| | or if they will serve as a subinvestigator under another | |
| | study investigator. Considerations that may be | |
| | presented to the local physician in making this | |
| | decision include, but are not limited to, the following: | |
| | Both roles are responsible for protocol and GCP | |
| | training and compliance, but the principal | |
| | investigator is also responsible for reporting to the | |
| | IRB in accordance with 21CFR56. | |
| | The principal investigator is responsible for records | |
| | maintenance, including archiving. | |
| | The principal investigator is ultimately responsible | |
| | for all investigator obligations under 21CFR312. | |
| | Note that existing investigators on the study may | |
| | not be willing to accept a remote subinvestigator. | |
| | If the local physician is willing to accept the principal | |
| | investigator responsibilities under 21CFR312, skip to | |
| | Section B. If the local physician is not willing to | |
| | accept the principal investigator responsibilities under | |
| | 21CFR312, proceed with step A.3. | |
| A.3 | Contact the sponsor to request activation as a | NM |
| | subinvestigator under an existing principal investigator. | |
| A.4 | Upon confirmation from the sponsor that an existing | NM |
| | investigator is willing to work with the local physician | |
| | as a subinvestigator, provide the recommended | |
| | subinvestigator budget to the local physician for review | |
| | and provide the XXX “CTA Amendment Language | |
| | Template”, XXX “Subinvestigator Contract Template”, | |
| | and Subinvestigator Budget to the sponsor for | |
| | negotiation with the principal investigator. See Section | |
| | C for the process of drafting and negotiating the | |
| | budget. | |
| A.5 | Confirm with the sponsor contact if OncoBay templates | NM |
| | for Financial Disclosure (SA001_T08) and Site | |
| | Delegation of Authority Log (CM001_T06) will be | |
| | accepted. If they are not accepted, obtain the required | |
| | templates from the sponsor contact. | |
| A.6 | Collect the financial disclosure form, CV, medical | NM |
| | license, and delegation of authority from the local | |
| | physician and review in accordance with SA001_ | |
| | WP06 Collection and Processing of Essential | |
| | Documents. Upon receipt and execution of the Short | |
| | Form Contract, forward to the sponsor contact and | |
| | advise the sponsor contact that they or their delegate | |
| | will need to collect an updated 1572 from the | |
| | principal investigator with this subinvestigator added. | |
| A.7 | Collect evidence of GCP training from the local | NM |
| | physician. Upon receipt, forward to the sponsor | |
| | contact. Reference SOP XXX “Preparing a Local Site | |
| | for Trial Participation” for the process of provisioning | |
| | GCP training if the site is not able to provide this. | |
| A.8 | Request confirmation of all required regulatory | NM |
| | documents from the sponsor contact on XXX “Receipt | |
| | and Activation Form”. | |

Activating a Local Oncologist as the Principal Investigator

| | | Respon- |
|---|---|---|
| | | sible |
| Step | Action | Party |
| B.1 | Upon confirmation that the local physician is willing to | NM |
| | accept the principal investigator responsibilities under | |
| | 21CFR312, provide the recommended principal | |
| | investigator budget to the local physician for review | |
| | with the XXX “Simplified Contract”. See Section C for | |
| | the process of drafting and finalizing the budget and | |
| | Clinical Trial Agreement (CTA). | |
| B.2 | Confirm with the sponsor contact if OncoBay templates | NM |
| | for Authorization for Regulatory Green Light (SA001_ | |
| | F10 and all templates/forms referenced therein) will | |
| | be accepted. If they are not accepted, obtain the | |
| | templates from the sponsor contact. At a minimum, | |
| | obtain the final approved protocol and master informed | |
| | consent from the sponsor contact. | |
| B.4 | Collect all documents required for regulatory green | NM |
| | light from the local physician and review in accordance | |
| | with SA001_WP001 Submissions to Regulatory | |
| | Authorities and IEC-IRB and SA001_WP06 | |
| | Collection and Processing of Essential Documents. | |
| B.5 | Upon receipt and execution of the “Short Form | NM |
| | Contract”, forward the Authorization for Regulatory | |
| | Green Light and the associated completed package | |
| | to the sponsor contact. | |
| B.6 | Confirm with the sponsor contact if OncoBay | NM |
| | templates for Site Activation Form (SA002_F02 and | |
| | all templates/forms referenced therein) will be | |
| | accepted. If they are not accepted, obtain the templates | |
| | from the sponsor contact. | |
| B.7 | Confirm with the sponsor contact any study-specific | NM |
| | documents that are required for Investigational Produce | |
| | (IP) release. | |
| B.8 | Collect all documents required for IP release from the | NM |
| | local physician and review in accordance with SA002 | |
| | Site Activation. | |
| B.9 | Forward the Site Activation Form (SA002_F02) and | NM |
| | the associated completed package to the sponsor | |
| | contact. | |

Drafting and Finalizing a Budget and Contract

| | | Responsible |
|---|---|---|
| Step | Action | Party |
| C.1 | Draft the sponsor and site study budgets in | NM |
| | accordance with the schedule of assessments in | |
| | the protocol using the OncoBay Coverage | |
| | Analysis database. If the local physician will be a | |
| | subinvestigator, remove all personnel fees and | |
| | present these separately to the sponsor as a | |
| | recommendation for reimbursement to the | |
| | principal investigator. | |
| | Note: these budgets are not negotiable and the | |
| | process for budget review is presumed to end | |
| | here. | |
| C.2 | Notify legal@oncobay.com if the local physician | NM |
| | will serve as subinvestigator or principal | |
| | investigator. | |
| C.3 | If the local physician will be a subinvestigator, | Legal |
| | draft XXX “CTA Amendment Language” and | Representative |
| | XXX “Subinvestigator Contract”. If the local | |
| | physician will be the investigator, draft the XXX | |
| | “Simplified Contract”. | |
| C.4 | Provide the appropriate documents to the NM for | Legal |
| | provision to the sponsor contact or site as | Representative |
| | appropriate. If the local physician will be a | |
| | subinvestigator, the process stops here. If the | |
| | local physician will be a principal investigator, | |
| | proceed to step C.5. | |
| C.5 | Negotiate and finalize the XXX “Simplified | Legal |
| | Contract” with the physician in accordance with | Representative |
| | SOP “Contract Negotiation”. | |

### Example 3: Standard Operation Procedure for Preparing a Local Site for Trial Participation

Example 3 describes an exemplary Standard Operating Procedure (SOP) for use in preparing a local site for trial participation. The standard operating procedure outlines the various steps required to execute the method described herein for preparing a local site for trial participation.

| Document | | | | Department/ | Central Project |
|---|---|---|---|---|---|
| Type: | Standard Operating Procedure | | | Owner: | Operations |
| Title: | Preparing a Local Site for | | | Document | xx |
| | Trial Participation | | | Number: | |
| Review | Every 2 | Effective | DD-Mon- | Version | 1.0 |
| Frequency: | years | Date: | YYYY | Number: | |

### Purpose

This Standard Operating Procedure (SOP) describes the process of preparing the local physician and the clinic for research qualification for the purpose of creating a personal clinical trial environment at a location where a patient is already receiving care.

### Scope

This SOP applies to all OncoBay employees and contractors involved in preparing a local physician for personal clinical trial environments. Vendors qualified for any of the services described in this SOP by QA011 Selection and Qualification of Contract Service Providers will follow their own processes for provisioning equipment, staff, or technology.

Preparing the clinic for research qualification comprises complying with 21 CFR § 312 and the International Council for Harmonization for Good Clinical Practices (ICH E6) and comprises:
obtaining regulatory approvals;
providing training to staff, and potentially providing remote staff if needed;
creating standard operating procedures for the clinic to maintain research qualification;
providing an electronic Investigator Site File;
obtaining equipment; or
obtaining technology to enable a remote clinical trial.

Technology provided to the local physician may include telemedicine, cloud computing, video conferencing, electronic learning management systems, electronic signature software, data privacy and security software. All technology vendors must be qualified under QA011 Selection and Qualification of Contract Service Providers.

### Definitions and Abbreviations

| Term | Definition |
|---|---|
| Network | This is a designated role that can be performed by a Director, |
| Manager | Manager, Lead, or Specialist within Network Operations at |
| | OncoBay Clinical. |

### Procedure

GCP Training Provisioning

| | | Responsible |
|---|---|---|
| Step | Action | Party |
| A.1 | Request evidence of Good Clinical Practice (GCP) | Network |
| | training in compliance with SA001_WP06 | Manager |
| | Collection and Processing of Essential Documents. | (NM) |
| A.2 | If the site is unable to produce acceptable evidence | NM |
| | of GCP training, contact QA@oncobay.com to | |
| | request links for the following CITI courses to be | |
| | sent to the relevant contacts at the site: | |
| | GCP - all staff on Site Delegation of Authority | |
| | Log | |
| | Clinical Research: An Introduction - all staff on | |
| | Site Delegation of Authority Log | |
| | CRC - staff delegated study coordinator | |
| | responsibilities on the Site Delegation of | |
| | Authority Log | |
| | If the site produces acceptable evidence of GCP | |
| | training from all staff listed on the Site Delegation | |
| | of Authority Log, contact QA@oncobay.com to | |
| | request links for the following CITI courses to be | |
| | sent to the relevant contacts at the site: | |
| | Clinical Research: An Introduction - all staff on | |
| | Site Delegation of Authority Log | |
| | CRC - staff delegated study coordinator | |
| | responsibilities on the Site Delegation of | |
| | LAuthority og | |
| A.3 | Distribute requested CITI training links to site as | Quality |
| | required. | Assurance |
| | | Representative |
| A.4 | Request printed certifications of training | NM |
| | completion from the site, and process in | |
| | accordance with SA001_WP06. Instruct the site | |
| | to prioritize GCP training as this certificate is | |
| | required for regulatory green light. | |
| A.5 | Provide copies of all certifications of training | NM |
| | received in the investigator site file. | |

Site Evaluation

| | | Responsible |
|---|---|---|
| Step | Action | Party |
| B.1 | Schedule the site evaluation with the investigator(s) | NM |
| | and appropriate study site personnel (e.g., study | |
| | coordinator, research nurse, pharmacist, and lab | |
| | personnel), allowing sufficient time for the | |
| | assessment. | |
| B.2 | Reference the Network Evaluation Assessment | NM |
| | annotated report if required and ask open-ended | |
| | questions to be able to identify any potential issues at | |
| | the site that is being assessed. | |
| B.3 | Discuss the role of OncoBay Clinical as an agent and | NM |
| | representative of the sponsor. | |
| B.4 | Review the site’s processes for conducting clinical | NM |
| | trials in accordance with ICH GCP guidelines. | |
| | Reference Section C to confirm adequacy of site’s | |
| | processes. | |
| B.5 | Verify that the studies take place in appropriate | NM |
| | clinical facilities and will be conducted by | |
| | and medical staff who have the appropriate training | |
| | investigators and experience to conduct trials in the | |
| | protocol specific disease. Reference Sections D, E, | |
| | and F to confirm adequacy of site’s facilities and | |
| | experience. | |
| B.6 | Assess how the site will manage source | NM |
| | adocumentation t the site. Reference CM001_F06 | |
| | Source Data Information Sheet. | |
| B.7 | Based on the discussions and any gaps noted, discuss | NM |
| | with the site whether or not the investigator plans to | |
| | delegate any study activities to OncoBay Clinical or | |
| | other third parties. If yes, verify that the site has SOPs | |
| | or equivalent processes in place to ensure appropriate | |
| | oversight with the involvement of third parties. | |

Site SOPs Gap Analysis

| | | Responsible |
|---|---|---|
| Step | Action | Party |
| C.1 | Request a list of clinical research SOPs from the site. | NM |
| C.2 | Verify that SOPs documenting the following | NM |
| | processes are available at the site: | |
| | Control of Investigational Drug, including Drug | |
| | Accountability | |
| | Investigator Record Keeping and Retention, | |
| | including Maintenance of Source Documentation | |
| | and Data Entry into Case Report Forms | |
| | Investigator Reports, specifically Safety Reporting | |
| | Assurance of IRB Review | |
| | Inspection of Investigator’s Records and Reports | |
| | Informed Consent | |
| C.3 | Perform a gap analysis to ensure the suitability of | NM |
| | available SOPs for conducting clinical trials in | |
| | accordance with ICH GCP guidelines. Ensure that | |
| | SOPs consider any services, facilities, or equipment | |
| | provided to the site by OncoBay or Vendors to ensure | |
| | that SOPs will cover the processes intended to be | |
| | used on this clinical trial. | |
| C.4 | Provide results of gap analysis to the site. Confirm if | NM |
| | the site will revise their SOPs or implement new | |
| | SOPs to close the gap. If the site will revise the SOPs, | |
| | proceed to step C.6. If the site will implement new | |
| | SOPs, proceed to step C.5. | |
| C.5 | Provide template SOPs to the site to address any gaps | NM |
| | identified. Request that the site review and revise as | |
| | necessary. | |
| C.6 | Collect updated SOPs from the site and confirm | NM |
| | suitability of available for conducting clinical trials in | |
| | accordance with ICH GCP guidelines. If gaps are | |
| | identified, return to step C.4. | |

Facilities and Equipment Gap Analysis

| | | Responsible |
|---|---|---|
| Step | Action | Party |
| D.1 | Request a list of required facilities and equipment | NM |
| | from the sponsor contact. Equipment commonly | |
| | required includes the following: | |
| | ECG machine | |
| | Infusion area with infusion chairs and pumps | |
| | Electrocardiogram | |
| | Locked drug cabinet | |
| | Chemotherapy preparation hood | |
| | Refrigerator | |
| | Freezer (−20° C. or −80° C.) | |
| D.2 | During the site evaluation, confirm the availability | NM |
| | and suitability of required facilities with the site. | |
| D.3 | Provide results of gap analysis to the site. Confirm if | NM |
| | the site will obtain access to appropriate facilities and | |
| | equipment or utilize an OncoBay vendor to close the | |
| | gap. If the site will obtain access to facilities and | |
| | equipment, proceed to step D.5. If the site will utilize | |
| | an OncoBay vendor, proceed to step D.4. | |
| D.4 | Provide equipment to site as requested and proceed to | NM |
| | step D.5. Equipment should be purchased for the site | |
| | to expedite provision. If the sponsor already has a | |
| | rental vendor set-up, equipment may be rented. | |
| D.5 | Confirm with the site that they have obtained access | NM |
| | to appropriate facilities and equipment and confirm | |
| | suitability of available facilities and equipment for | |
| | conducting the clinical trial. If gaps are identified, | |
| | return to step D.3. | |

Staff Capabilities Gap Analysis

| | | Responsible |
|---|---|---|
| Step | Action | Party |
| E.1 | Request a list of any specific staff experience and | NM |
| | expertise capabilities from the sponsor contact. | |
| E.2 | During the site evaluation, confirm the suitability of | NM |
| | experience and expertise of assigned staff with the | |
| | site. | |
| E.3 | Provide results of gap analysis to the site. Confirm if | NM |
| | the site will obtain access to appropriate training or | |
| | additional resources or utilize OncoBay services to | |
| | close the gap. If the site will obtain access to | |
| | appropriate training or additional resources, proceed | |
| | to step E.5. If the site will utilize OncoBay services, | |
| | proceed to step E.4. | |
| E.4 | Implement services required in accordance with | NM |
| | XXX “SOP Management of Patient Data in Real | |
| | Time” and proceed to step E.5. | |
| E.5 | Confirm with the site that they have obtained access | NM |
| | to appropriate training or additional resources | |
| | confirm suitability of available staff for conducting | |
| | the clinical trial. If gaps are identified, return to step | |
| | E.3. | |

Telemedicine Requirements Analysis

| | | Responsible |
|---|---|---|
| Step | Action | Party |
| F.1 | During the site evaluation, confirm site’s needs for | NM |
| | support with patient management on the clinical trial, | |
| | including the informed consent process. | |
| F.2 | Implement services required in accordance with | NM |
| | XXX “SOP Management of Patient Data in Real | |
| | Time”. | |

Investigator Site File Provisioning

| | | Responsible |
|---|---|---|
| Step | Action | Party |
| G.1 | During the site evaluation, confirm site’s access to an | NM |
| | acceptable electronic Investigator Site File (elSF) | |
| | system. | |
| G.2 | Provide results of gap analysis to the site. Confirm if | NM |
| | the site will obtain access to an appropriate elSF or | |
| | utilize OncoBay recommended services to close the | |
| | gap. If the site will obtain access to an appropriate | |
| | elSF,proceed to step G.3. If the site will utilize | |
| | OncoBay recommended services, proceed to step | |
| | G.4. | |
| G.3 | Confirm suitability of elSF for retaining records in | NM |
| | accordance with ICH GCP guidelines. If the elSF is | |
| | not suitable, return to step G.2; otherwise proceed to | |
| | step G.5. | |
| G.4 | Provide the site with an appropriate link to Veeva | NM |
| | SiteVault (e.g. https://sites.veeva.com/sitevault-free- | |
| | sign-up/site-type/) and proceed to step G.5. | |
| G.5 | After receipt of regulatory green light, and in | NM |
| | advance of site activation, provide the ISF to the site | |
| | in accordance with SA002 WP01 Preparing the | |
| | Investigator Site File. | |
| G.6 | Request from the sponsor contact that all study | NM |
| | specific documents are sent to the site for the ISF. | |
| G.7 | Instruct the site to upload all documents received | NM |
| | from OncoBay and the sponsor to the ISF. | |

### Example 4: Standard Operating Procedure for Management of Patient Data in Real Time

Example 4 describes an exemplary Standard Operating Procedure (SOP) for use in management of patient data collected in real time. The standard operating procedure outlines the various steps required to execute the method described herein for preparing a local site for trial participation.

| Document | | | | Department/ | Central Project |
|---|---|---|---|---|---|
| Type: | Standard Operating Procedure | | | Owner: | Operations |
| Title: | Management of Patient | | | Document | xx |
| Review | Data in Real Time | | | Number: | |
| Frequency: | Every 2 | Effective | DD-Mon- | Version | 1.0 |
| | years | Date: | YYYY | Number: | |

### Purpose

This Standard Operating Procedure (SOP) describes the process of facilitating conduct of the clinical trial for a local physician for the purpose of creating a personal clinical trial environment at a location where the patient is already receiving care.

### Scope

This SOP applies to all OncoBay employees and contractors involved in facilitating conduct of personal clinical trial environments. Vendors qualified for any of the services described by this SOP by QA011 Selection and Qualification of Contract Service Providers will follow their own processes for building infrastructure.

Facilitating conduct of the clinical trial for the local physician includes the following tasks:
Preparing subject level specific training for the site and visit-by-visit walk-through guides for the purposes of ensuring just-in-time training for the local physician and local clinic personnel, as well as for ensuring appropriate documentation of procedures conducted for the purposes of ensuring ongoing compliance with ICH E6 (R2) Sections 4.1.2 and 4.1.5;
Providing ongoing remote support for the local physician;
Facilitating data entry, answering study-related questions, regulatory document management, and trial management to ensure continued compliance with 21 CFR § 312 and the International Council for Harmonization for Good Clinical Practices (ICH E6).
Closure of the clinic and return of equipment or technology when the patient completes the trial or trial follow-up, or discontinues the trial.

### Definitions and Abbreviations

| Term | Definition |
|---|---|
| Study Visit | Outlines created by visit with instructions for the site |
| Walk-throughs | from the protocol, and data fields available for entry |
| | ensuring the collection of appropriate source |
| | documentation and collection of data required for the |
| | Case Report Form (CRF). |
| Network | This is a designated role that can be performed by a |
| Manager | Director, Manager, Lead, or Specialist within Network |
| | Operations at OncoBay Clinical. |

### Procedure

Preparing & Provisioning Subject Level Specific Training and Documentation

| | | Responsible |
|---|---|---|
| Step | Action | Party |
| A.1 | Upon site activation, request creation of study visit | Network |
| | walk-throughs in Genius My Trial Navigator. | Manager |
| | | (NM) |
| A.2 | Create the study visit walk-throughs in accordance | Data |
| | with XXX “SOPEDCBuild”. The study visit walk- | Manager |
| | throughs should meet the following minimum | |
| | criteria: | |
| | Go-live of all visits should be targeted at least 14 | |
| | days in advance of each visit. Visits prior to first | |
| | dose may go-live with less time between go-live | |
| | and anticipated subject visit. | |
| | Appropriate instructions for the site should be | |
| | included in an appropriate order according to the | |
| | protocol. Instructions should be taken directly | |
| | from the protocol as much as possible. | |
| | Data fields should be presented in the order of the | |
| | instructions, and must ensure the collection of the | |
| | minimum data required for appropriate source | |
| | documentation and collection of all fields required | |
| | by the CRF. | |
| A.3 | Conduct system training with the investigators, | Data |
| | study coordinators, and data entry staff for the site | Manager |
| | in accordance with the Site Delegation of Authority | |
| | Log. Issue a training log to the site and instruct them | |
| | to file it in their ISF. | |

Set-Up and Execution of Ongoing Remote Support

| | | Responsible |
|---|---|---|
| Step | Action | Party |
| B.1 | If the site has confirmed a need for support with | NM |
| | informed consent or patient management, request | |
| | activation of telemedicine in Genius ENGAGE | |
| | (elCF) and/or Genius ROSA (Telemedicine). | |
| | Identify in the request if patient management | |
| | support, informed consent support, or both is | |
| | required from the site. | |
| B.2 | Create the study build in accordance with XXX | Data |
| | “SOPBuild”. The telemedicine should meet the | Manager |
| | following criteria: | |
| | Go-live at least one day in advance of first subject | |
| | visit. | |
| | Upload of current IRB-approved informed consent, | |
| | if informed consent support is required. | |
| B.3 | Conduct system training with the investigators and | Data |
| | study coordinators for the site in accordance with the | Manager |
| | Site Delegation of Authority Log. Issue a training | |
| | log to the site and instruct them to file it in their ISF. | |
| B.4 | Provide copies of relevant site SOPs to OncoBay | NM |
| | team members expected to perform services, | |
| | including but not limited to, the Medical Director, | |
| | Study Coordinator, Data Entry team, and Regulatory | |
| | Document team. | |
| B.5 | Review site SOPs for services to be performed and | Project |
| | document training in accordance with PM003_WP02 | Team |
| | Project-Specific Training. | |
| B.6 | Follow site SOPs for services to be performed. | Project |
| | | Team |

Clinic Closure at End of Study

| | | Responsible |
|---|---|---|
| Step | Action | Party |
| C.1 | After the patient completes the trial, confirm if the | NM |
| | investigator is willing to remain active on the trial. | |
| | If the investigator is not willing to remain active, | |
| | skip to step C.3. | |
| C.2 | If the investigator is willing to remain active on the | NM |
| | study despite completion of the patient, contact the | |
| | sponsor contact to confirm if they will proceed with | |
| | the site remaining active. | |
| C.3 | Upon notification from the sponsor or investigator | NM |
| | that the site will be closed, proceed with the | |
| | following: | |
| | Request from Data Management deactivation of | |
| | systems utilized in accordance with XXX | |
| | “SOPSystemClose”. | |
| | Work with relevant vendors to ensure return of | |
| | OncoBay-provided equipment and facilities as | |
| | required by contract | |
| | Notify internal resource managers of expected | |
| | project end date | |
| | Ensure that appropriate Oncobay-provided team | |
| | members are in attendance at the Site Close-out | |
| | Visit on the site’s behalf | |
| C.4 | Follow up with the investigator after the close-out | NM |
| | visit to ensure that the investigator understands | |
| | ongoing obligations under ICH GCP. | |

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

What is claimed:

1. A method of creating a remote clinical trial to recruit one or more patients to an existing clinical trial comprising:
a) identifying one or more patients;
b) identifying one or more local physicians having a clinic local to the one or more patients;
c) preparing the one or more local physicians and the respective clinic for research qualification; and
d) facilitating conduct of the clinical trial for the one or more local physicians.

2. The method of claim 1, wherein identifying one or more patients comprises matching each patient's diagnosis and treatment requirements to a clinical trial.

3. The method of claim 2, further comprising qualifying each patient for a clinical trial.

4. The method of claim 1, wherein identifying one or more local physicians comprises:
a) selecting each patient's personal physician to become the local physician;
b) providing each patient with technology and resources to identify the local physician who is not the patient's personal physician; or
c) providing each patient's personal physician with technology and resources to identify the local physician.

5. The method of claim 4, wherein identifying one or more local physicians further comprises approving and qualifying at least one local physician to serve as an Investigator of the clinical trial by a Sponsor of the clinical trial.

6. The method of claim 5, wherein approving and qualifying the at least one local physician comprises complying with 21 CFR § 312 and the International Council for Harmonization for Better Health/Good Clinical Practices (ICH E6 (R2)).¹ ¹ 21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

7. The method of claim 1, wherein preparing the at least one local physician comprises executing a contract with each local physician to serve as a Principal Investigator or a sub-Investigator for the remote clinical trial.

8. The method of claim 6, wherein the contract for the Principal Investigator comprises a trial budget comprising an amount of payment to the local physician and a budget for support services for the local physician.

9. The method of claim 6, wherein the contract for the sub-Investigator comprises a trial budget comprising an amount of payment to the local physician, services a Principal Investigator will provide to the local physician, services the local physician will provide, and a budget for support services for the local physician.

10. The method of claim 1, wherein preparing the clinic for research qualification comprises complying with 21 CFR § 312 and the International Council for Harmonization for Better Health/Good Clinical Practices (ICH E6) and comprises:
a) obtaining regulatory approvals;
b) providing training to staff;
c) creating standard operating procedures for the clinic to maintain research qualification;
d) providing an electronic Investigator Site File;
e) obtaining equipment; or
f) obtaining technology to enable a remote clinical trial.² ² 21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

11. The method of claim 10, wherein the regulatory approvals comprise collecting and maintaining regulatory documents.

12. The method of claim 11, wherein the regulatory documents are transmitted or delivered to the regulatory authority.

13. The method of claim 10, wherein staff may be on site at the clinic or may work remotely.

14. The method of claim 10, wherein the technology comprises one or more of telemedicine, cloud computing, video conferencing, electronic learning management systems, electronic signature software, data privacy and security software.

15. The method of claim 1, wherein facilitating conduct of the clinical trial for the local physician comprises preparing subject level specific training for the site and visit-by-visit walk-through guides for the purposes of ensuring just-in-time training for the local physician and local clinic personnel, as well as for ensuring appropriate documentation of procedures conducted for the purposes of ensuring ongoing compliance with ICH E6 (R2) Sections 4.1.2 and 4.1.5.³

16. The method of claim 14, wherein facilitating conduct of the clinical trial for the local physician further comprises providing ongoing remote support for the local physician.

17. The method of claim 14, wherein facilitating conduct of the clinical trial for the local physician further comprises one or more of facilitating data entry, answering study-related questions, regulatory document management, and trial management to ensure continued compliance with 21 CFR § 312 and the International Council for Harmonization for Better Health/Good Clinical Practices (ICH E6).⁴ ^{3,4}21 CFR § 312 is herein incorporated by reference with regard to procedures governing the use of investigational new products including the submission to, and review by, the US FDA; and the ICH (E6) is herein incorporated by reference with regard to Good Clinical Practice procedures, guidelines and standards for clinical trials.

18. The method of claim 1, wherein facilitating conduct of the clinical trial for the local physician comprises one or more of ongoing facilitation of clinical site activities conducting the trial until the patient completes the trial or trial follow-up, or discontinues the trial.

19. The method of claim 18, wherein completion or discontinuation of the trial, comprises one or more of facilitating closure of the clinic and return of equipment or technology.
